(19) United States
(12) Patent Application Publication
(10) Pub. No.: US 2017/0029760 A1
(43) Pub. Date: Feb. 2, 2017

(54) FULL-AUTOMATIC MICROORGANISM DETECTING ENRICHMENT SYSTEM AND ENRICHMENT METHOD THEREOF

(71) Applicants: Gang NIU, (US); Huashan WANG, (US)

(72) Inventor: Gang NIU, Beijing (CN)

(73) Assignees: Gang NIU, Beijing (CN); Huashan WANG, Tianjin (CN)

(21) Appl. No.: 15/106,930

(22) PCT Filed: Dec. 31, 2014
(86) PCT No.: PCT/CN2014/095848

§ 371 Date: Jun. 21, 2016

(30) Foreign Application Priority Data
Dec. 31, 2013 (CN) 201310751864.2

(51) Int. Cl. C12M 1/26; C12M 1/34; C12M 3/00; C12M 1/12; C12M 1/00; C12M 1/36

(52) U.S. Cl. CPC C12M 33/14; C12M 29/04; C12M 33/18; C12M 41/48; C12M 23/44; C12M 37/00; C12M 41/12

(57) ABSTRACT

A full-automatic microorganism detecting enrichment system and the enrichment method thereof are provided. A transmission apparatus is controlled by a industrial control computer to transmit a to-be-detected sample placed on a preset operating position, a culture medium comprising culture, a pipeline filter specially for collecting bacteria and a filter head sequentially into a sterilization cabin, an middle package removing cabin, an inner package removing cabin, an enrichment operating cabin, a buffer cabin and a positive bacteria filling cabin to perform corresponding operations and enrich the microorganism in the to-be-detected samples. Said system can achieve a fully automated enrichment of the microorganism in the to-be-detected samples, needs no manual operation, and saves time and labor force. Further, it can avoid false positive or false negative result caused by an effect of human factor, and achieve sterile and automatic operation with an accurate detection result.

### TECHNICAL FIELD

The present invention relates to the technical field of microorganism detection. More particularly, the invention relates to a full-automatic microorganism detecting enrichment system and the enrichment method thereof for implementing the enrichment process in the detection of microorganisms.

### BACKGROUND ART

At present, all microorganism detecting enrichments almost are conducted manually. The units for sterilizing, bacteria enrichment driving, filling the culture medium and positive bacteria filling are independent of each other, and the environments for filling positive bacteria and collecting bacteria are different, which means the whole process needs to be conducted in the different environments, and transmitting the to-be-detected samples between different environments has to rely on manual operation, thus it is easy to be influenced by human factors, which might prone to produce false positive or false negative results and affect the accuracy and timeliness of detection.

With the increase of people's requirements for food and drug safety, a full-automatic microorganism enrichment system without human intervention is an inevitable demand for food and drug safety.

### CONTENTS OF INVENTION

The aim of the present invention is providing a full-automatic microorganism detecting enrichment system and the enrichment method thereof, which can realize an automated enrichment of the to-be-detected samples, effectively avoid false positive or false negative results caused by an effect of human factors, and achieve an accurate detection result.

To reach above aims, the present invention employs following solutions.

A full-automatic microorganism detecting enrichment system comprises:

a preset operating position, for placing to-be-detected samples, a culture medium containing culture, a pipeline filter specially for collecting bacteria and a filter head; the filter utilizing a filter film for filtering the to-be-detected samples and enriching the microorganisms possibly contained in the to-be-detected samples;

a sterilization cabin for conducting sterilization, a middle package removing cabin for removing the middle package, an inner package removing cabin for removing the inner package, an enrichment operating cabin for injecting the to-be-detected samples into a filter to filter the enriched microorganism, enclosing the filter bottom by a filter head, and selectively injecting the culture into the filter, a buffer cabin for cutting the filter pipeline after the enrichment, and a positive bacteria filling cabin for filling the positive bacteria, being disposed adjacent successively behind the preset operating position; above said cabins being connected and separated respectively by a cabin separating mechanism;

a transmitting apparatus, for transmitting the to-be-detected samples placed on the preset operating position, the culture medium, the filter and the filter head into each corresponding cabin respectively, the transmitting apparatus starting from the preset operating position and running through above said cabins; and

a industrial control computer, for electrically connecting above each cabin, each cabin separating mechanism and the transmitting apparatus.

In a preferred embodiment, a fixing device is provided on the transmitting apparatus for fixing the to-be-detected samples, the culture medium containing the culture, the filter and the filter head, and/or

the transmitting apparatus could be divided into a to-be-detected sample transmitting apparatus, a culture medium transmitting apparatus, a filter transmitting apparatus and a filter head transmitting apparatus; and/or

position sensors are provided on the preset positions of the transmitting apparatus within each cabin and electrically connected with the industrial control computer; and/or

the transmitting apparatus is a belt conveyor or a roller transmitting apparatus; the belt or roller could be driven by rotating magnetic fluid within each cabin actuated by a motor outside each cabin, or directly driven by a motor provided inside of each cabin; and/or

each cabin separating mechanism is a separating valve.

In a preferred embodiment, an air pressure regulating device and a ventilation device are provided outside of each cabin to adjust the cabin pressure; each air pressure regulating device and each ventilation device electrically connect to said industrial control computer respectively; each air pressure regulating device contains a pressure sensor for detecting the pressure of each cabin, and each pressure sensor electrically connects to the industrial control computer; and/or

sterilization devices are respectively arranged outside said sterilization cabin, the enrichment operating cabin, and the buffer cabin for sterilizing corresponding cabins.

In a preferred embodiment, a middle package removing device and an inner package removing device, which are used for removing the middle package and inner package of the filter and the filter head, are respectively arranged above said transmitting apparatus within said middle package removing cabin and said inner package removing cabin; the inner package removing device and the middle package removing device are electrically connected with the industrial control computer respectively.

In a preferred embodiment, the pipeline filters are three parallel tanks with outlets on both ends, each tank has filter film on the bottom, after being filled with the to-be-detected samples, microorganisms are enriched by the filter film; the pipelines of filter are three parallel pipelines, one end of which is provided on the top of said filter and communicated with each tank, and the other end of which is a filter needle; said filter has a set position in the enrichment operating cabin, and the set position is where the bottom of each tank locates in the supporting hole of the waste liquid trough;

an enrichment operation manipulator is arranged above said transmitting apparatus in the enrichment operating cabin, the enrichment operation manipulator inserts the filter needle into the to-be-detected samples and replaces the filter needle with a culture needle, then inserting it into the culture of the culture medium; the enrichment operation manipulator can make the filter shift between the set position and the transmitting apparatus; an enrichment driving mechanism is provided on one side of the enrichment operation manipulator in the proximity of said buffer cabin for driving the to-be-detected items or culture to be filled into three parallel pipelines and to enter into said filter;

a packaging mechanism is provided near said set position in the enrichment operating cabin for packaging the bottom of each tank by the filter head after the process of filtering and enriching;

the enrichment operation manipulator, the enrichment driving mechanism and the packaging mechanism are electrically connected to the industrial control computer respectively.

In a preferred embodiment, said enrichment driving mechanism is a peristaltic pump, a first bracket and a second bracket are provided on the pump head of the peristaltic pump for clamping the three parallel pipelines when the enrichment operation manipulator holds the filter needle to insert into the to-be-detected samples, the first bracket is retractably fixed on one side of the peristaltic pump head near the enrichment operation manipulator, the second bracket is provided on the pump head of the peristaltic pump retractably and rotatably; the second bracket has a first position and a second position, on the first position, the second bracket is arranged side by side at the side of the first bracket for clamping the three parallel pipelines together with the first bracket, on the second position, the second bracket rotates to a location where the three parallel pipelines can be correspondingly placed into the pump head of the peristaltic pump; when operating, the first bracket and the second bracket clamp the three parallel pipelines respectively, then the second bracket is rotated to the second position and the first and second brackets retract back, thereby putting the three parallel pipelines into the pump head of the peristaltic pump

said enrichment operation manipulator further includes a culture medium heat-seal mechanism for sealing at most two pipelines of the three parallel pipelines before the culture medium entering into said filter after that said filter finished filtering and enriching the to-be-detected samples, thereby the culture medium could be selectively injected into the microorganism enriched filters;

a triple pressure sensor is provided in the enrichment operating cabin to detect the inner pressure of said filter;

the peristaltic pump, the first bracket, the second bracket, the culture medium heat-seal mechanism and the triplet pressure sensor are electrically connected with the industrial control computer, respectively.

In a preferred embodiment, a pipeline cutting mechanism are provided above the transmitting apparatus in said buffer cabin, and

the pipeline cutting mechanism is electrically connected with the industrial control computer.

In a preferred embodiment, said positive bacteria filling cabin has a heating function, a positive bacteria filling mechanism is provided above the transmitting apparatus in the positive bacteria filling cabin; a temperature control apparatus is further provided in the positive bacteria filling cabin;

the positive bacteria filling mechanism and the temperature control apparatus are electrically connected with the industrial control computer, respectively; and/or

the industrial control computer is a form of the host computer, and each cabin has separate control unit.

A detection method applied to any above full-automatic microorganism detecting enrichment system, comprises following steps of:

the first step: placing to-be-detected samples, the culture medium containing culture, the pipeline filter specially for collecting bacteria and the filter head on the transmitting apparatus of the preset operating position;

the second step: opening the cabin separating mechanism between the preset operating position and the sterilization cabin, transmitting the to-be-detected samples, the culture medium containing culture, the pipeline filter specially for collecting bacteria and the filter head into said sterilization cabin, closing the cabin separating mechanisms on both sides of the sterilization cabin and carrying out sterilization, then ventilating for balancing the pressure in the sterilization cabin after finishing the sterilization;

the third step: opening the cabin separating mechanism between the sterilization cabin and the middle package removing cabin, transmitting the to-be-detected samples, the culture medium containing culture, the pipeline filter specially for collecting bacteria and the filter head into the middle package removing cabin, closing the cabin separating mechanisms on both sides of the middle package removing cabin, and removing the middle packages of the filter and the filter head;

the fourth step: opening the cabin separating mechanism between the middle package removing cabin and the inner package removing cabin, transmitting the to-be-detected samples, the culture medium containing culture, the pipeline filter specially for collecting bacteria and the filter head into the inner package removing cabin, closing the cabin separating mechanisms on both sides of inner package removing cabin, and removing the inner package of the filter and the filter head;

the fifth step: opening the cabin separating mechanism between the inner package removing cabin and the enrichment operating cabin, transmitting the to-be-detected samples, the culture medium containing culture, the pipeline filter specially for collecting bacteria and the filter head into the enrichment operating cabin, closing the cabin separating mechanisms on both sides of the enrichment operating cabin, filtering and enriching the microorganisms contained in the to-be-detected samples in the filter, enclosing the filter bottom by the filter head, then selectively injecting the culture into the filter, placing the enriched and enclosed filter on the transmitting apparatus, obtaining appropriate number of the enriched and enclosed filters;

the sixth step: opening the cabin separating mechanism between the enrichment operating cabin and the buffer cabin, transmitting the enriched and enclosed filters into the buffer cabin, closing the cabin separating mechanisms on both sides of buffer cabin, cutting the pipeline of enriched and enclosed filters; and

the seventh step, opening the cabin separating mechanism between the buffer cabin and the positive bacteria filling cabin, transmitting the enriched and enclosed filters into the positive bacteria filling cabin, closing the cabin separating mechanisms on both sides of the positive bacteria filling cabin, filling the positive bacteria into the enriched and enclosed filters after finishing the pipeline cutting, then completing the microorganism enrichment of the detected samples.

In a preferred embodiment, each time the cabin separating mechanism between the preset operating position, the sterilization cabin, the middle package removing cabin, the inner package removing cabin and the enrichment operating cabin open, the pressure of the former cabin is lower than that of the latter cabin, to guarantee no bacteria being taken into the enrichment operating cabin; and each time the cabin separating mechanisms between the enrichment operating cabin, the buffer cabin and the positive bacteria filling cabin open, the pressure of the former cabin is higher than that of the latter cabin, to guarantee no positive bacteria being adversely transferred into the enrichment operating cabin; and/or

in said seventh step, after closing the cabin separating mechanisms on both sides of the positive bacteria filling cabin, a further step is included: sterilizing the buffer cabin; and/or

said filters are three parallel tanks with outlets on both ends, the culture are selectively injected into at most two of those tanks; and/or

in said fifth step, used samples and culture medium are transferred back to the inner package removing cabin by the transmitting apparatus for recycling.

The beneficial effects of the present invention are as follows. The present invention provides a full-automatic microorganism detecting enrichment system and the enrichment method, which can achieve a fully automated enrichment of the microorganism in the to-be-detected samples, needs no manual operation, and saves time and labor force. Further, it can effectively avoid false positive or false negative result caused by an effect of human factors, and achieve sterile and automatic operation with an accurate detecting result. The enrichment process is less error-prone and is able to be operated continuously, with no need to pause and sterile the enrichment operating cabin after stopping the enrichment operation, consequently it has a better implementation effect.

### DESCRIPTION OF DRAWINGS

FIG. 1 is a schematic top view for a preferred embodiment of a full-automatic microorganism detecting enrichment system in the present invention.

FIG. 2 is a schematic main view for a preferred embodiment of a full-automatic microorganism detecting enrichment system in the present invention.

FIG. 3 is a flow chart for a preferred embodiment of a full-automatic microorganism detecting enrichment system in the present invention.

### REFERENCE SIGNS IN FIGURES

101 preset operation position
102 sterilization cabin
103 middle package removing cabin
104 inner package removing cabin
105 enrichment operating cabin
106 buffer cabin
107 positive bacteria filling cabin
110 air pressure regulating device
120 sterilization device
130 ventilation device
200 position sensor
310 middle package removing device
320 inner package removing device
140 cabin separating mechanism
400 transmitting apparatus
500 enrichment driving mechanism
510 enrichment operation manipulator
520 pipeline cutting mechanism
530 packaging mechanism
700 positive bacteria filling mechanism
800 industrial control computer
900 triplet pressure sensor
1000 to-be-detected samples
1100 filter

### DESCRIPTION OF EMBODIMENTS

First of all, it needs to be declared that following description is only used for explaining the possible embodiments of the present invention, however, the description should not be considered as a limit to the protection scope of the present invention.

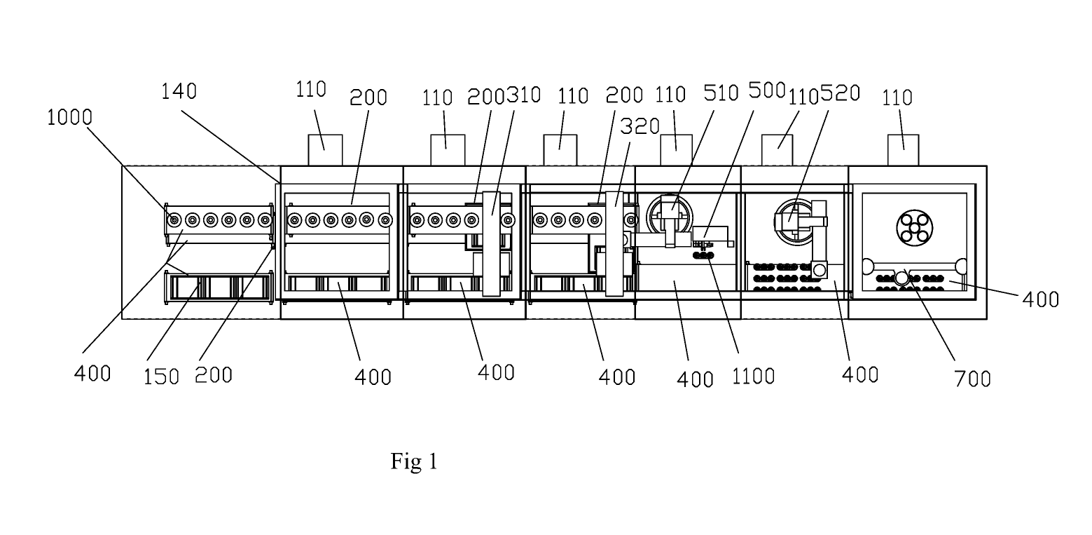

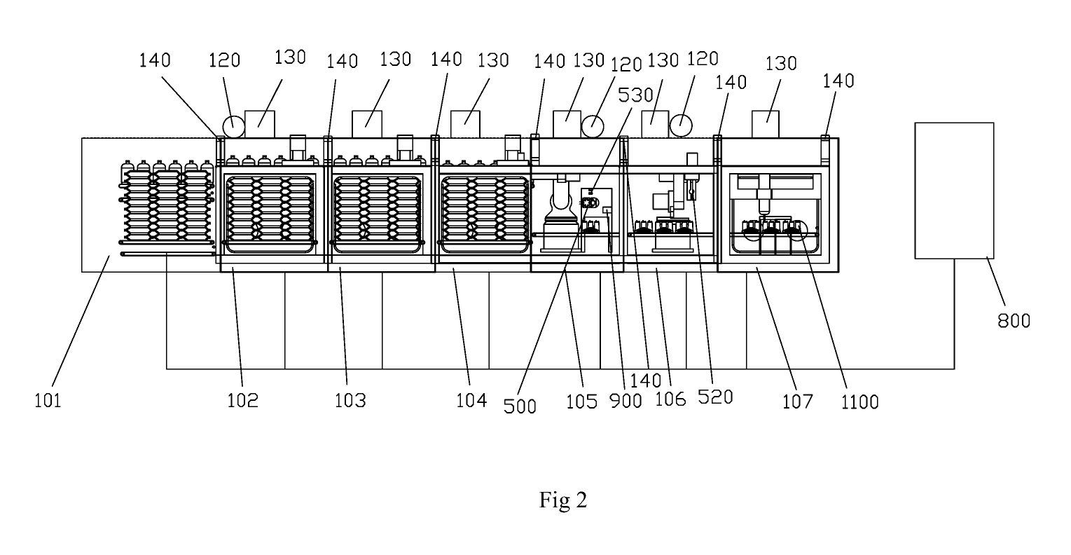

As shown in FIGS. 1 and 2, the present invention provides a full-automatic microorganism detecting enrichment system, comprising:

A preset operating position 101, behind which there are disposed adjacent successively by a sterilization cabin 102 for conducting sterilization, a middle package removing cabin 103 for removing the middle package, an inner package removing cabin 104 for removing the inner package, an enrichment operating cabin 105 for injecting the to-be-detected samples into a filter to filter the enriched microorganism, enclosing the filter bottom by a filter head, and selectively injecting the culture into the filter, a buffer cabin 106 for cutting the filter pipeline after the enrichment, and a positive bacteria filling cabin 107 for filling the positive bacteria; said cabins being connected and separated respectively by a cabin separating mechanism 140; a transmitting apparatus 400 and a industrial control computer 800.

The preset operating position 101 is used for placing to-be-detected samples 1000, a culture medium containing culture, a pipeline filter 1100 specially for collecting bacteria and a filter head. Said filter utilizes a filter film for filtering the to-be-detected samples and enriching the microorganisms possibly contained in the to-be-detected samples.

The transmitting apparatus 400 is used for transmitting the to-be-detected samples placed on the preset operating position 101, the culture medium, the filter 1100 and the filter head into said each corresponding cabin respectively. The transmitting apparatus 400 starts from the preset operating position and runs through above cabins.

The industrial control computer 800 is electrically connected with said cabins, each cabin separating mechanism 140 and the transmitting apparatus 400 to control said cabins performing corresponding actions.

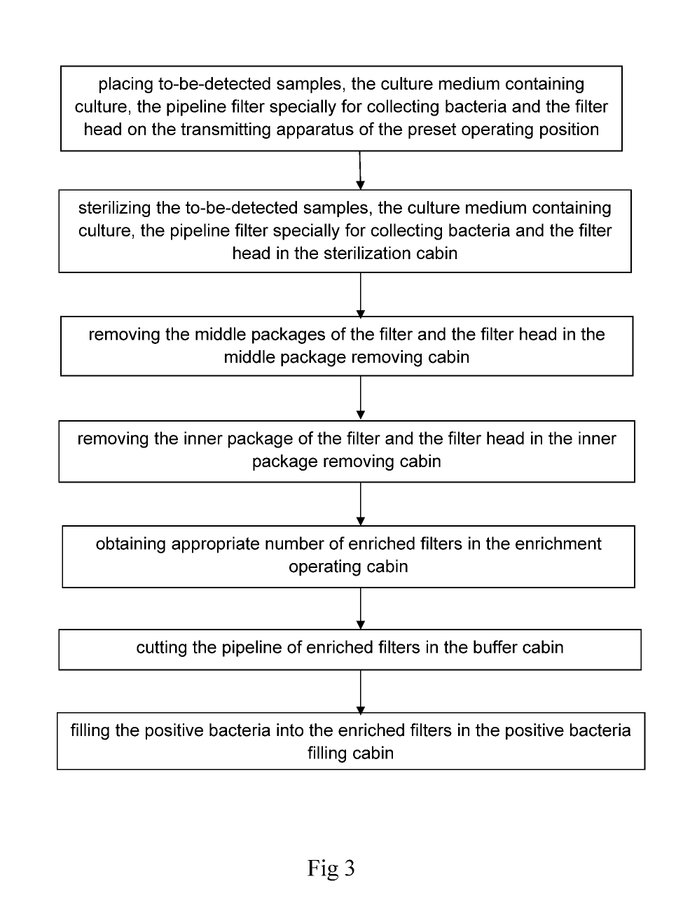

As shown in FIG. 3, the present invention further provides a full-automatic microorganism enriching method, which comprises following steps of:

(i) first, placing to-be-detected samples 1000, the culture medium containing culture, the pipeline filter 1100 specially for collecting bacteria and the filter head on the transmitting apparatus 400 of the preset operating position;

(ii) second, opening the cabin separating mechanism 140 between the preset operating position 101 and the sterilization cabin 102, transmitting the to-be-detected samples 1000, the culture medium containing culture, the pipeline filter 1100 specially for collecting bacteria and the filter head into the sterilization cabin 102, closing the cabin separating mechanisms 140 on both sides of the sterilization cabin 102 and carrying out sterilization, then ventilating for balancing the pressure in the sterilization cabin 102 after finishing the sterilization;

(iii) third, opening the cabin separating mechanism between the sterilization cabin 102 and the middle package removing cabin 103, transmitting the to-be-detected samples 1000, the culture medium containing culture, the pipeline filter 1100 specially for collecting bacteria and the filter head into the middle package removing cabin 103, closing the cabin separating mechanisms 140 on both sides of the middle package removing cabin 103, and removing the middle packages of the filter 1100 and the filter head;

(iv) fourth, opening the cabin separating mechanism 140 between the middle package removing cabin 103 and the inner package removing cabin 104, transmitting the to-be-detected samples 1000, the culture medium containing culture, the pipeline filter 1100 specially for collecting bacteria and the filter head into the inner package removing cabin 104, closing the cabin separating mechanisms 140 on both sides of inner package removing cabin 104, and removing the inner package of the filter 1100 and the filter head;

(v) fifth, opening the cabin separating mechanism 140 between the inner package removing cabin 104 and the enrichment operating cabin 105, transmitting the to-be-detected samples 1000, the culture medium containing culture, the pipeline filter 1100 specially for collecting bacteria and the filter head into the enrichment operating cabin 105, closing the cabin separating mechanisms 140 on both sides of the enrichment operating cabin 105, filtering and enriching the microorganisms contained in the to-be-detected samples 1000 in the filter 1100, enclosing the filter bottom by the filter head, then selectively injecting the culture into the filter 1100, placing the enriched and enclosed filter 1100 on the transmitting apparatus, obtaining appropriate number of the enriched and enclosed filters;

(vi) sixth, opening the cabin separating mechanism 140 between the enrichment operating cabin 105 and the buffer cabin 106, transmitting the enriched and enclosed filters 1100 into the buffer cabin 106, closing the cabin separating mechanisms 140 on both sides of buffer cabin 105, cutting the pipeline of enriched and enclosed filters 1100;

(vii) seventh, opening the cabin separating mechanism 140 between the buffer cabin 106 and the positive bacteria filling cabin 107, transmitting the enriched and enclosed filters 1100 into the positive bacteria filling cabin 107, closing the cabin separating mechanisms 140 on both sides of the positive bacteria filling cabin 107, filling the positive bacteria into the enriched and enclosed filters 1100 after finishing the pipeline cutting, then completing the microorganism enrichment of the detected samples.

To sum up, the present invention is suitable for the enrichment required by the microorganism limit test in the field of food and drug safety. The whole process is controlled by the industrial control computer 800, thus it achieves the automation and eliminates the false positive or false negative results possibly caused by human factors.

In a preferred embodiment of the present invention, as shown in FIGS. 1 and 2, a fixing device 150 is provided on the transmitting apparatus 400 for fixing the to-be-detected samples 1000, the culture medium containing the culture, the filter 1100 and the filter head, in order to ensure that the samples 1000, the culture medium containing the culture, the filter 1100 and the filter head would not be misplaced or dumped in the process of conveying due to inertia, which will cause contaminations within each cabin and affect the accuracy of the operation in each cabin.

The transmitting apparatus 400 could be divided into a to-be-detected sample transmitting apparatus, a culture medium transmitting apparatus, a filter transmitting apparatus and a filter head transmitting apparatus, in order to transmit the corresponding items and lower the complexity of the transmitting apparatus 400, for example, it only needs to provide a filter transmitting apparatus in the buffer cabin 106 and positive bacteria filling cabin 107, and doesn't need other three type of transmitting apparatus. Thus it can reduce the space and save the cost.

Position sensors 200 are provided on the preset positions of the transmitting apparatus 400 within each cabin. Each position sensor 200 is electrically connected with the industrial control computer 800, so as to ensure the accurate operation positions of corresponding items within each cabin during the conveying process of the transmitting apparatus 400. Preferably, each position sensor 200 is an infrared sensor or a radio sensor.

The cabin separating mechanism 140 is a separating valve.

In a preferred embodiment, the transmitting apparatus 400 is a belt conveyor or a roller transmitting apparatus. The belt or roller could be driven by rotating magnetic fluid within each cabin actuated by a motor outside each cabin, or directly driven by a motor provided inside of each cabin.

In a preferred embodiment, as shown in FIGS. 1 and 2, an air pressure regulating device 110 and a ventilation device 130 are provided outside of each cabin to adjust the cabin pressure. Each air pressure regulating device 110 and each ventilation device 130 electrically connect to said industrial control computer respectively. Each air pressure regulating device 110 contains a pressure sensor for detecting the pressure of each cabin. Each pressure sensor electrically connects to the industrial control computer. Therefore, the pressure of each cabin can be controlled by the air pressure regulating device 110 and ventilation device 130 during the transfer between cabin and cabin. Sterilization devices 120 are respectively arranged outside said sterilization cabin 102, the enrichment operating cabin 105, and the buffer cabin 106 for sterilizing corresponding cabins. Thus, each cabin could be sterilized to avoid contamination.

Preferably, in the full-automatic microorganism enrichment method of the present preferred embodiment, each time the cabin separating mechanisms 140 between the preset operating position 101, the sterilization cabin 102, the middle package removing cabin 103, the inner package removing cabin 104 and the enrichment operating cabin 105 open, the pressure of the former cabin is lower than that of the latter cabin, to guarantee no bacteria being taken into the enrichment operating cabin 105. Each time the cabin separating mechanisms 140 between the enrichment operating cabin 105, the buffer cabin 106 and the positive bacteria filling cabin 107 open, the pressure of the former cabin is higher than that of the latter cabin, to guarantee no positive bacteria being adversely transferred into the enrichment operating cabin 106.

Furthermore, in the seventh step, after closing the cabin separating mechanisms 140 on both sides of the positive bacteria filling cabin 106, a further step is included, i.e. to sterilize the buffer cabin 106. The buffer cabin 106 is mainly used to transfer the positive bacteria and prevent the positive bacteria from mutual infection. This is because if the enrichment operating cabin 105 is directly connected to the positive bacteria filling cabin 106, the positive bacteria may enter into the enrichment operating cabin 105. However, since the enrichment operating cabin 105 operates continuously, keeping sterilizing it will be a waste of time. Therefore, the buffer cabin 106 could connect to the enrichment operating cabin 105 after being sterilized and the operation of the enrichment operating cabin 105 can not be interrupted. When the buffer cabin 106 is isolated with the enrichment operating cabin 105, it can be communicated with the positive bacteria filling cabin 107. After the enriched and enclosed filters 1100 are cut and transferred into the positive bacteria filling cabin 106, the buffer cabin 106 is capable of being isolated and sterilized, then it can be communicated with the enrichment operating cabin 105 to do the next transfer of the filters 1100. Therefore, it is capable of avoiding the possibility of the positive bacteria in the positive bacteria filling cabin 107 entering into the enrichment operating cabin 107, and the operation of the enrichment operating cabin 105 could not be interrupted, so that the whole system has good working continuity and high efficiency.

As shown in FIGS. 1 and 2, in a preferred embodiment of the present invention, a middle package removing device 310 and an inner package removing device 320, which are used for removing the middle package and inner package of the filter and the filter head, are respectively arranged above said transmitting apparatus 400 within said middle package removing cabin 103 and said inner package removing cabin 104. The middle package removing device 310 and inner package removing device 320 are electrically connected with the industrial control computer respectively.

In a preferred embodiment of the present invention, as shown in FIGS. 1 and 2, the pipeline filters 1100 are three parallel tanks with outlets on both ends, preferably the tanks are transparent. Each tank has filter film on the bottom. After being filled with the to-be-detected samples, microorganisms are enriched by the filter film. The pipelines of filter 1100 are three parallel pipelines, one end of which is provided on the top of said filter 1100 and communicated with each tank, and the other end of which is a filter needle. Said filter 1100 has a set position in the enrichment operating cabin 105, and the set position is where the bottom of each tank locates in the supporting hole of the waste liquid trough.

An enrichment operation manipulator 510 is arranged above said transmitting apparatus 400 in the enrichment operating cabin 105. The enrichment operation manipulator 510 inserts the filter needle into the to-be-detected samples and replaces the filter needle with a culture needle, then inserting it into the culture of the culture medium. The enrichment operation manipulator 510 can make the filter 1100 shift between the set position and the transmitting apparatus. An enrichment driving mechanism 500 is provided on one side of the enrichment operation manipulator 510 in the proximity of said buffer cabin for driving the to-be-detected items or culture to be filled into three parallel pipelines and enter into said filter 1100.

A packaging mechanism 530 is provided near said set position in the enrichment operating cabin 105 for packaging the bottom of each tank by the filter head after the process of filtering and enriching.

The enrichment operation manipulator 510, the enrichment driving mechanism 500 and the packaging mechanism 530 are electrically connected to the industrial control computer 800 respectively and controlled by it.

In a preferred embodiment of the present invention, said enrichment driving mechanism 500 is a peristaltic pump. A first bracket and a second bracket are provided on the pump head of the peristaltic pump for clamping the three parallel pipelines when the enrichment operation manipulator holds the filter needle to insert into the to-be-detected samples. Moreover, the first bracket is retractably fixed on one side of the peristaltic pump head near the enrichment operation manipulator 510, and the second bracket is provided on the pump head of the peristaltic pump retractably and rotatably. The second bracket has a first position and a second position. On the first position, the second bracket is arranged side by side at the side of the first bracket for clamping the three parallel pipelines together with the first bracket. On the second position, the second bracket rotates to a location where the three parallel pipelines can be correspondingly placed into the head of the peristaltic pump. When operating, the first bracket and the second bracket clamp the three parallel pipelines respectively, then the second bracket is rotated to the second position and the first and second brackets retract back, thereby putting the three parallel pipelines into the pump head of the peristaltic pump.

Said enrichment operation manipulator 510 further includes a culture medium heat-seal mechanism for sealing at most two pipelines of the three parallel pipelines before the culture medium entering into said filter after that said filter finished filtering and enriching the to-be-detected samples. Thus, the culture medium could be selectively injected into the microorganism enriched filters. Optionally, the three parallel pipelines could be heat-sealed one, or two or none of them.

A triple pressure sensor 900 is provided in the enrichment operating cabin 105 to detect the inner pressure of said filter 1100, in order to modulate the pressure of the enrichment operating cabin by detecting the pressure in the filter.

Said peristaltic pump, the first bracket, the second bracket, the culture medium heat-seal mechanism and the triplet pressure sensor 900 are electrically connected with the industrial control computer 800, respectively.

Therefore, the operation in said enrichment operating cabin 105 comprises following steps:

Said filter 1100 is placed on the preset position by the enrichment operation manipulator 510, and then the filter needle is inserted into the to-be-detected samples. The three parallel pipelines are placed into the pump head of the peristaltic pump by the first and second brackets. The peristaltic pump starts to work, the filter 1100 filters the fluid of the to-be-detected samples 1000 and the possible microorganisms are enriched on the filter film. Then the enrichment operation is completed. After that, the packaging mechanism 530 encloses the bottom of each tank by the filter head to complete the packaging operation. Moreover, one pipeline is selected to be closed by the culture medium heat-seal mechanism. The enrichment operation manipulator 510 replaces the filter needle with a culture needle and inserts it into the culture of the culture medium. The culture is injected into the two pipelines by the peristaltic pump and enriched in two corresponding tanks. Above all, enrichment and packaging of the filter and injection of the culture are completed in the enrichment operating cabin 105.

The industry control computer performs internal control programs according to the signals detected by each sensor to respectively control the pressure in each cabin, the transmission of the transmitting apparatus and corresponding actions of actuators in each cabin accordingly. The control programs belong to the common knowledge known by the skilled in the art, which are not created by the present inventor and therefore will not be described in detail here.

Accordingly, in one preferred embodiment of the full-automatic microorganism enrichment method of the present invention, said filters are three parallel tanks, in two of which the culture could be filled in.

Preferably, in said fifth step, used samples and culture medium are transferred back to the inner package removing cabin by the transmitting apparatus 400 for recycling.

Preferably, as shown by FIGS. 1 and 2, in one preferred embodiment of the present invention, a pipeline cutting mechanism 520 are provided above the transmitting apparatus 400 in said buffer cabin 106, in order to package the pipeline of the filter.

The pipeline cutting mechanism 520 is electrically connected with the industrial control computer 800.

Preferably, as depicted in FIGS. 1 and 2, in one preferred embodiment of the present invention, said positive bacteria filling cabin 107 has a heating function, so that the positive bacteria can be cultured in the positive bacteria filling cabin 107 immediately after the completion of the positive bacteria filling. A positive bacteria filling mechanism 700 is provided above the transmitting apparatus 400 in the positive bacteria filling cabin 107. A temperature control apparatus is further provided in the positive bacteria filling cabin.

The positive bacteria filling mechanism 700 and the temperature control apparatus are electrically connected with the industrial control computer 800, respectively.

Preferably, the industrial control computer 800 could be a form of the host computer. Each cabin may have separate control unit.

In summary, the present invention provides a full-automatic microorganism detecting enrichment system and the enrichment method, which can achieve a fully automated enrichment of the microorganism in the to-be-detected samples, needs no manual operation, and saves time and labor force. Further, it can effectively avoid false positive or false negative result caused by an effect of human factors, and achieve sterile and automatic operation with an accurate detecting result. The enrichment process is less error-prone and is able to be operated continuously, with no need to pause and sterile the enrichment operating cabin after stopping the enrichment operation, consequently it has a better implementation effect.

1. A full-automatic microorganism detecting enrichment system, wherein the system comprises:
a preset operating position, for placing to-be-detected samples, a culture medium containing culture, a pipeline filter specially for collecting bacteria and a filter head; the filter utilizing a filter film for filtering the to-be-detected samples and enriching the microorganisms possibly contained in the to-be-detected samples;
a sterilization cabin for conducting sterilization, a middle package removing cabin for removing the middle package, an inner package removing cabin for removing the inner package, an enrichment operating cabin for injecting the to-be-detected samples into a filter to filter the enriched microorganism, enclosing the filter bottom by a filter head, and selectively injecting the culture into the filter, a buffer cabin for cutting the filter pipeline after the enrichment, and a positive bacteria filling cabin for filling the positive bacteria, being disposed adjacent successively behind the preset operating position; above said cabins being connected and separated respectively by a cabin separating mechanism;
a transmitting apparatus, for transmitting the to-be-detected samples placed on the preset operating position, the culture medium, the filter and the filter head into each corresponding cabin respectively, the transmitting apparatus starting from the preset operating position and running through above said cabins; and
a industrial control computer, for electrically connecting above each cabin, each cabin separating mechanism and the transmitting apparatus.

2. The full-automatic microorganism detecting enrichment system according to claim 1, wherein a fixing device is provided on the transmitting apparatus for fixing the to-be-detected samples, the culture medium containing the culture, the filter and the filter head, and/or
the transmitting apparatus could be divided into a to-be-detected sample transmitting apparatus, a culture medium transmitting apparatus, a filter transmitting apparatus and a filter head transmitting apparatus; and/or
position sensors are provided on the preset positions of the transmitting apparatus within each cabin and electrically connected with the industrial control computer; and/or
the transmitting apparatus is a belt conveyor or a roller transmitting apparatus; the belt or roller could be driven by rotating magnetic fluid within each cabin actuated by a motor outside each cabin, or directly driven by a motor provided inside of each cabin; and/or
each cabin separating mechanism is a separating valve.

3. The full-automatic microorganism detecting enrichment system according to claim 1, wherein an air pressure regulating device and a ventilation device are provided outside of each cabin to adjust the cabin pressure; each air pressure regulating device and each ventilation device electrically connect to said industrial control computer respectively; each air pressure regulating device contains a pressure sensor for detecting the pressure of each cabin, and each pressure sensor electrically connects to the industrial control computer; and/or
sterilization devices are respectively arranged outside said sterilization cabin, the enrichment operating cabin, and the buffer cabin for sterilizing corresponding cabins.

4. The full-automatic microorganism detecting enrichment system according to claim 1, wherein a middle package removing device and an inner package removing device, which are used for removing the middle package and inner package of the filter and the filter head, are respectively arranged above said transmitting apparatus within said middle package removing cabin and said inner package removing cabin; the inner package removing device and the middle package removing device are electrically connected with the industrial control computer respectively

5. The full-automatic microorganism detecting enrichment system according to claim 1, wherein the pipeline filters are three parallel tanks with outlets on both ends, each tank has filter film on the bottom, after being filled with the to-be-detected samples, microorganisms are enriched by the filter film; the pipelines of filter are three parallel pipelines, one end of which is provided on the top of said filter and communicated with each tank, and the other end of which is a filter needle; said filter has a set position in the enrichment operating cabin, and the set position is where the bottom of each tank locates in the supporting hole of the waste liquid trough;
an enrichment operation manipulator is arranged above said transmitting apparatus in the enrichment operating cabin, the enrichment operation manipulator inserts the filter needle into the to-be-detected samples and replaces the filter needle with a culture needle, then inserting it into the culture of the culture medium; the enrichment operation manipulator can make the filter shift between the set position and the transmitting apparatus; an enrichment driving mechanism is provided on one side of the enrichment operation manipulator in the proximity of said buffer cabin for driving the to-be-detected items or culture to be filled into three parallel pipelines and to enter into said filter;
a packaging mechanism is provided near said set position in the enrichment operating cabin for packaging the bottom of each tank by the filter head after the process of filtering and enriching;
the enrichment operation manipulator, the enrichment driving mechanism and the packaging mechanism are electrically connected to the industrial control computer respectively.

6. The full-automatic microorganism detecting enrichment system according to claim 5, wherein said enrichment driving mechanism is a peristaltic pump, a first bracket and a second bracket are provided on the pump head of the peristaltic pump for clamping the three parallel pipelines when the enrichment operation manipulator holds the filter needle to insert into the to-be-detected samples, the first bracket is retractably fixed on one side of the peristaltic pump head near the enrichment operation manipulator, the second bracket is provided on the pump head of the peristaltic pump retractably and rotatably; the second bracket has a first position and a second position, on the first position, the second bracket is arranged side by side at the side of the first bracket for clamping the three parallel pipelines together with the first bracket, on the second position, the second bracket rotates to a location where the three parallel pipelines can be correspondingly placed into the pump head of the peristaltic pump; when operating, the first bracket and the second bracket clamp the three parallel pipelines respectively, then the second bracket is rotated to the second position and the first and second brackets retract back, thereby putting the three parallel pipelines into the pump head of the peristaltic pump
said enrichment operation manipulator further includes a culture medium heat-seal mechanism for sealing at most two pipelines of the three parallel pipelines before the culture medium entering into said filter after that said filter finished filtering and enriching the to-be-detected samples, thereby the culture medium could be selectively injected into the microorganism enriched filters;
a triple pressure sensor is provided in the enrichment operating cabin to detect the inner pressure of said filter;
the peristaltic pump, the first bracket, the second bracket, the culture medium heat-seal mechanism and the triplet pressure sensor are electrically connected with the industrial control computer, respectively.

7. The full-automatic microorganism detecting enrichment system according to claim 1, wherein a pipeline cutting mechanism are provided above the transmitting apparatus in said buffer cabin, and
the pipeline cutting mechanism is electrically connected with the industrial control computer.

8. The full-automatic microorganism detecting enrichment system according to claim 1, wherein said positive bacteria filling cabin has a heating function, a positive bacteria filling mechanism is provided above the transmitting apparatus in the positive bacteria filling cabin; a temperature control apparatus is further provided in the positive bacteria filling cabin;
the positive bacteria filling mechanism and the temperature control apparatus are electrically connected with the industrial control computer, respectively; and/or
the industrial control computer is a form of the host computer, and each cabin has separate control unit.

9. A full-automatic microorganism enriching method comprises following steps of:
the first step: placing to-be-detected samples, the culture medium containing culture, the pipeline filter specially for collecting bacteria and the filter head on the transmitting apparatus of the preset operating position;
the second step: opening the cabin separating mechanism between the preset operating position and the sterilization cabin, transmitting the to-be-detected samples, the culture medium containing culture, the pipeline filter specially for collecting bacteria and the filter head into said sterilization cabin, closing the cabin separating mechanisms on both sides of the sterilization cabin and carrying out sterilization, then ventilating for balancing the pressure in the sterilization cabin after finishing the sterilization;
the third step: opening the cabin separating mechanism between the sterilization cabin and the middle package removing cabin, transmitting the to-be-detected samples, the culture medium containing culture, the pipeline filter specially for collecting bacteria and the filter head into the middle package removing cabin, closing the cabin separating mechanisms on both sides of the middle package removing cabin, and removing the middle packages of the filter and the filter head;
the fourth step: opening the cabin separating mechanism between the middle package removing cabin and the inner package removing cabin, transmitting the to-be-detected samples, the culture medium containing culture, the pipeline filter specially for collecting bacteria and the filter head into the inner package removing cabin, closing the cabin separating mechanisms on both sides of inner package removing cabin, and removing the inner package of the filter and the filter head;
the fifth step: opening the cabin separating mechanism between the inner package removing cabin and the enrichment operating cabin, transmitting the to-be-detected samples, the culture medium containing culture, the pipeline filter specially for collecting bacteria and the filter head into the enrichment operating cabin, closing the cabin separating mechanisms on both sides of the enrichment operating cabin, filtering and enriching the microorganisms contained in the to-be-detected samples in the filter, enclosing the filter bottom by the filter head, then selectively injecting the culture into the filter, placing the enriched and enclosed filter on the transmitting apparatus, obtaining appropriate number of the enriched and enclosed filters;
the sixth step: opening the cabin separating mechanism between the enrichment operating cabin and the buffer cabin, transmitting the enriched and enclosed filters into the buffer cabin, closing the cabin separating mechanisms on both sides of buffer cabin, cutting the pipeline of enriched and enclosed filters; and
the seventh step, opening the cabin separating mechanism between the buffer cabin and the positive bacteria filling cabin, transmitting the enriched and enclosed filters into the positive bacteria filling cabin, closing the cabin separating mechanisms on both sides of the positive bacteria filling cabin, filling the positive bacteria into the enriched and enclosed filters after finishing the pipeline cutting, then completing the microorganism enrichment of the detected samples.

10. The full-automatic microorganism enriching method according to claim 9, wherein each time the cabin separating mechanism between the preset operating position, the sterilization cabin, the middle package removing cabin, the inner package removing cabin and the enrichment operating cabin open, the pressure of the former cabin is lower than that of the latter cabin, to guarantee no bacteria being taken into the enrichment operating cabin; and each time the cabin separating mechanisms between the enrichment operating cabin, the buffer cabin and the positive bacteria filling cabin open, the pressure of the former cabin is higher than that of the latter cabin, to guarantee no positive bacteria being adversely transferred into the enrichment operating cabin; and/or
in said seventh step, after closing the cabin separating mechanisms on both sides of the positive bacteria filling cabin, a further step is included: sterilizing the buffer cabin; and/or
said filters are three parallel tanks with outlets on both ends, the culture are selectively injected into at most two of those tanks; and/or
in said fifth step, used samples and culture medium are transferred back to the inner package removing cabin by the transmitting apparatus for recycling.
